(19) United States
(12) Patent Application Publication
(10) Pub. No.: US 2012/0191058 A1
(43) Pub. Date: Jul. 26, 2012

(54) Apparatus for Detecting Liquid Level, Remaining Liquid Quantity and Dripping Speed of Container

(75) Inventor: Yi-Wing Wu, Taipei City (TW)

(73) Assignees: ALLTEK MARINE ELECTRONICS CORP., Taipei City (TW); PANTEK TECHNOLOGY CORP., Taipei City (TW)

(21) Appl. No.: 13/012,120

(22) Filed: Jan. 24, 2011

(51) Int. Cl. A61M 5/172

(52) U.S. Cl. 604/404

(57) ABSTRACT

An apparatus includes a detecting electrode, a liquid sensing module, and a connecting means. The detecting electrode is attached to an outer surface of an intravenous drip container. The liquid sensing module includes a clock generator generating a clock signal, a waveform generating circuit coupled to the detecting electrode to generate an analog driving signal by using the clock signal, a grounding electrode grounded through the waveform generating circuit, an analog-to-digital converter converting the analog driving signal to a digital voltage signal, and a capacitance determining circuit receiving the digital voltage signal to determine a value of a capacitance of a capacitor formed by the detecting electrode and the grounding electrode. The connecting means connects the detecting electrode to the liquid sensing module.

### BACKGROUND OF THE INVENTION

1. Field of the Invention

The invention relates to an apparatus for detecting the liquid level, remaining liquid quantity, and dripping speed of an intravenous bag (or bottle or container).

2. Description of the Related Art

Supplying medicine and nutritional supplements to patients via drip injection is a very common medical treatment in a medical process in a hospital. However, during the drip injection process, health care workers and patients have to take note, at frequent intervals, of the height of the liquid in the dripping bottle. If health care workers and patients fail to notice that the liquid in the dripping bottle has been depleted, vascular obstruction or other emergency situations can result.

In view of this, at present, health care workers and patients can be warned by a suitable detecting device if the liquid in the dripping bottle is depleted or the height of liquid level in the dripping bottle is too low. Such a detecting device can monitor the drip injection process and thus reduce a load on health care workers and patients during the drip injection process and, accordingly, raise the quality of patient safety and health care.

Taiwan Patent No. M360703 discloses a detecting device, wherein the liquid level in the dripping bottle is detected by two infrared sensors. When the height of the liquid level is lower than a predetermined value, a warning message is sent to inform health care workers.

However, the detection of the liquid level in the dripping bottle by such a detecting device is not satisfactorily accurate. Furthermore, the detecting device can only detect the liquid level but cannot detect the remaining liquid quantity and the dripping speed of the dripping bottle, both of which are also important metrics for medical care.

### BRIEF SUMMARY OF THE INVENTION

The invention provides an apparatus for detecting a liquid level in an intravenous drip container. The apparatus in accordance with an exemplary embodiment of the invention includes a capacitor, a clock generator, a waveform generating circuit, an analog-to-digital converter, a capacitance determining circuit, and a processor. The capacitor includes a detecting electrode attached to an outer surface of the intravenous drip container, and a grounding electrode. The clock generator generates a clock signal. The waveform generating circuit is coupled to the detecting electrode to generate an analog driving signal by using the clock signal. The analog-to-digital converter converts the analog driving signal to a digital voltage signal. The capacitance determining circuit receives the digital voltage signal to determine a value of a capacitance of the capacitor. The processor converts the value of the capacitance into the liquid level in the intravenous drip container, and sends an alarm signal when the liquid level falls to a height of the detecting electrode. The grounding electrode is grounded through the waveform generating circuit and the processor.

In another exemplary embodiment, the grounding electrode is disposed adjacent to the waveform generating circuit.

In yet another exemplary embodiment, the processor includes a liquid level determining circuit configured to convert the value of the capacitance into the liquid level in the intravenous drip container, and a judgment circuit configured to judge the liquid level and send the alarm signal when the liquid level falls to the height of the detecting electrode.

In another exemplary embodiment, the apparatus further includes a digital-to-analog circuit, a power amplifier, a radio frequency transmitter, and an antenna, wherein the alarm signal is converted from digital to analog by the digital-to-analog circuit, amplified by the power amplifier, and sent out by the radio frequency transmitter and the antenna.

In yet another exemplary embodiment, the apparatus further includes an output terminal configured to send the alarm signal.

In another exemplary embodiment, the analog driving signal can be a sine, square or triangular waveform.

The invention also provides an apparatus for detecting a remaining liquid quantity in an intravenous drip container. The apparatus in accordance with an exemplary embodiment of the invention includes a capacitor, a clock generator, a waveform generating circuit, an analog-to-digital converter, a capacitance determining circuit, and a processor. The capacitor includes a detecting electrode attached to an outer surface of the intravenous drip container, and a grounding electrode. The clock generator generates a clock signal. The waveform generating circuit is coupled to the detecting electrode to generate an analog driving signal by using the clock signal. The analog-to-digital converter converts the analog driving signal to a digital voltage signal. The capacitance determining circuit receives the digital voltage signal to determine a value of a capacitance of the capacitor. The processor converts the value of the capacitance into the remaining liquid quantity in the intravenous drip container, judges the remaining liquid quantity, and sends an alarm signal when a liquid level in the intravenous drip container falls to a height of the detecting electrode. The grounding electrode is grounded through the waveform generating circuit and the processor.

In another exemplary embodiment, the grounding electrode is disposed adjacent to the waveform generating circuit.

In yet another exemplary embodiment, the processor includes a liquid quantity determining circuit configured to convert the value of the capacitance into the remaining liquid quantity in the intravenous drip container, and a judgment circuit configured to judge the remaining liquid quantity and sending out the alarm signal when the liquid level falls to the height of the detecting electrode.

In another exemplary embodiment, the liquid quantity determining circuit includes a lookup table of non-equidistant intervals, by which the remaining liquid quantity is determined.

In yet another exemplary embodiment, the apparatus further includes a digital-to-analog circuit, a power amplifier, a radio frequency transmitter, and an antenna, wherein the alarm signal is converted from digital to analog by the digital-to-analog circuit, amplified by the power amplifier, and sent out by the radio frequency transmitter and the antenna.

In another exemplary embodiment, the apparatus further includes an output terminal configured to send out the alarm signal.

In yet another exemplary embodiment, the analog driving signal can be a sine, square or triangular waveform.

The invention further provides an apparatus for detecting a remaining liquid quantity and dripping speed of an intravenous drip container. The apparatus in accordance with an exemplary embodiment of the invention includes a capacitor, a clock generator, a waveform generating circuit, an analog-to-digital converter, a capacitance determining circuit, and a processor. The capacitor includes a detecting electrode attached to an outer surface of the intravenous drip container, and a grounding electrode. The clock generator generates a clock signal. The waveform generating circuit is coupled to the detecting electrode to generate an analog driving signal by using the clock signal. The analog-to-digital converter converts the analog driving signal to a digital voltage signal. The capacitance determining circuit receives the digital voltage signal to determine a value of a capacitance of the capacitor. The processor converts the value of the capacitance into the remaining liquid quantity in the intravenous drip container, determines the dripping speed by variation of the remaining liquid quantity, judges the remaining liquid quantity, and sends an alarm signal when a liquid level in the intravenous drip container falls to a height of the detecting electrode. The grounding electrode is grounded through the waveform generating circuit and the processor.

In another exemplary embodiment, the grounding electrode is disposed adjacent to the waveform generating circuit.

In yet another exemplary embodiment, the processor includes a liquid quantity determining circuit configured to convert the value of the capacitance into the remaining liquid quantity in the intravenous drip container, a dripping speed determining circuit configured to determine the dripping speed by the variation of the remaining liquid quantity, and a judgment circuit configured to judge the remaining liquid quantity and send the alarm signal when the liquid level falls to the height of the detecting electrode.

In another exemplary embodiment, the liquid quantity determining circuit includes a lookup table of non-equidistant intervals, by which the remaining liquid quantity is determined.

In yet another exemplary embodiment, the apparatus further includes a digital-to-analog circuit, a power amplifier, a radio frequency transmitter, and an antenna, wherein the alarm signal is converted from digital to analog by the digital-to-analog circuit, amplified by the power amplifier, and sent out by the radio frequency transmitter and the antenna.

In another exemplary embodiment, the apparatus further includes an output terminal configured to send out the alarm signal.

In yet another exemplary embodiment, the analog driving signal can be a sine, square or triangular waveform.

The invention further provides an apparatus for detecting a liquid in an intravenous drip container. The apparatus in accordance with an exemplary embodiment of the invention includes a detecting electrode, a liquid sensing module, and a connecting means. The detecting electrode is attached to an outer surface of the intravenous drip container. The liquid sensing module includes a clock generator generating a clock signal, a waveform generating circuit coupled to the detecting electrode to generate an analog driving signal by using the clock signal, a grounding electrode grounded through the waveform generating circuit, an analog-to-digital converter configured to convert the analog driving signal to a digital voltage signal, and a capacitance determining circuit configured to receive the digital voltage signal to determine a value of a capacitance of a capacitor formed by the detecting electrode and the grounding electrode. The connecting means connects the detecting electrode to the liquid sensing module.

In another exemplary embodiment, the connecting means is a clip.

In yet another exemplary embodiment, the detecting electrode is T-shaped including a bottom portion attached to the outer surface of the intravenous drip container, and a top portion which is flexible to be held by the clip.

In another exemplary embodiment, the apparatus further includes an electrically isolated case in which the liquid sensing module is disposed.

In yet another exemplary embodiment, the apparatus further includes a processing and communication module configured to convert the value of the capacitance into a liquid level in the intravenous drip container, and to send an alarm signal when the liquid level falls to a height of the detecting electrode.

In another exemplary embodiment, the apparatus further includes a processing and communication module configured to convert the value of the capacitance into a remaining liquid quantity in the intravenous drip container, judge the remaining liquid quantity, and send an alarm signal when a liquid level in the intravenous drip container falls down to a height of the detecting electrode.

In yet another exemplary embodiment, the apparatus further includes a processing and communication module configured to convert the value of the capacitance into a remaining liquid quantity in the intravenous drip container, determine a dripping speed by variation of the remaining liquid quantity, judge the remaining liquid quantity, and send an alarm signal when a liquid level in the intravenous drip container falls to a height of the detecting electrode.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 depicts an intravenous dripping bag and a detecting apparatus in accordance with a first embodiment of the invention;

FIG. 2 is a schematic view of a detecting electrode of the detecting apparatus in accordance with the first embodiment of the invention;

FIG. 3 is a block diagram of the detecting apparatus in accordance with the first embodiment of the invention;

FIG. 4 is a block diagram of a detecting apparatus in accordance with a second embodiment of the invention;

FIG. 5 shows a relationship between the remaining liquid quantity in an intravenous drip bag and a voltage signal sent to a capacitance determining circuit of the detecting apparatus in accordance with the second embodiment of the invention;

FIG. 6 depicts an intravenous dripping bag and a measuring cylinder in accordance with a third embodiment of the invention;

FIG. 7 is a block diagram of a detecting apparatus in accordance with a third embodiment of the invention;

FIG. 8 is a block diagram of a detecting apparatus in accordance with a fourth embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

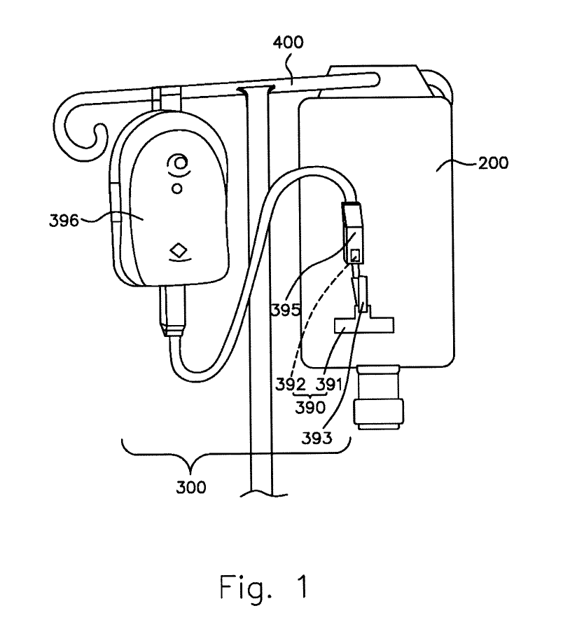

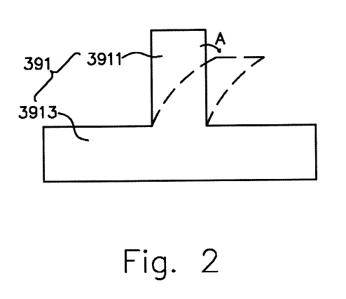

Referring to FIG. 1, in a first embodiment, a detecting apparatus 300 has a T-shaped detecting electrode 391, a connecting means (e.g. a clip) 393, an electrically isolated case 395, and a box 396. The box 396 and an intravenous drip bag (or bottle or container) 200 with a liquid (e.g. glucose solution) inside are supported by a drip stand 400. The intravenous drip bag 200 is marked on its lower portion to locate the detecting electrode 391. Referring to FIG. 2, the T-shaped detecting electrode 391 has a top portion 3911 and a bottom portion 3913. The bottom portion 3913 of the detecting electrode 391 is attached to an outer surface of the intravenous drip bag 200. The top portion 3911 of the detecting electrode 391 is flexible as indicated by an arrow A, to be held by the clip 393.

The detecting electrode 391 attached to the outer surface of the intravenous drip bag 200 and a grounding electrode 392 disposed in the electrically isolated case 395 serve as a capacitor 390. The value of the capacitance of the capacitor 390 changes due to variation of the liquid level in the intravenous drip bag 200. Therefore, the liquid level can be determined if the value of the capacitance of the capacitor 390 is obtained. The value of the capacitance C of the capacitor 390 can be obtained by the following formula:

C=(I×T)|V

wherein I is electric current, T is time, and V is voltage, all of which are measurable.

During operation, the detecting apparatus 300 keeps detecting the liquid level in the intravenous drip bag 200 by the detecting electrode 391. When the liquid level falls to the height of the detecting electrode 391, the detecting apparatus 300 emits an alarm signal via wire or wireless transmission, indicative of a need for medical care.

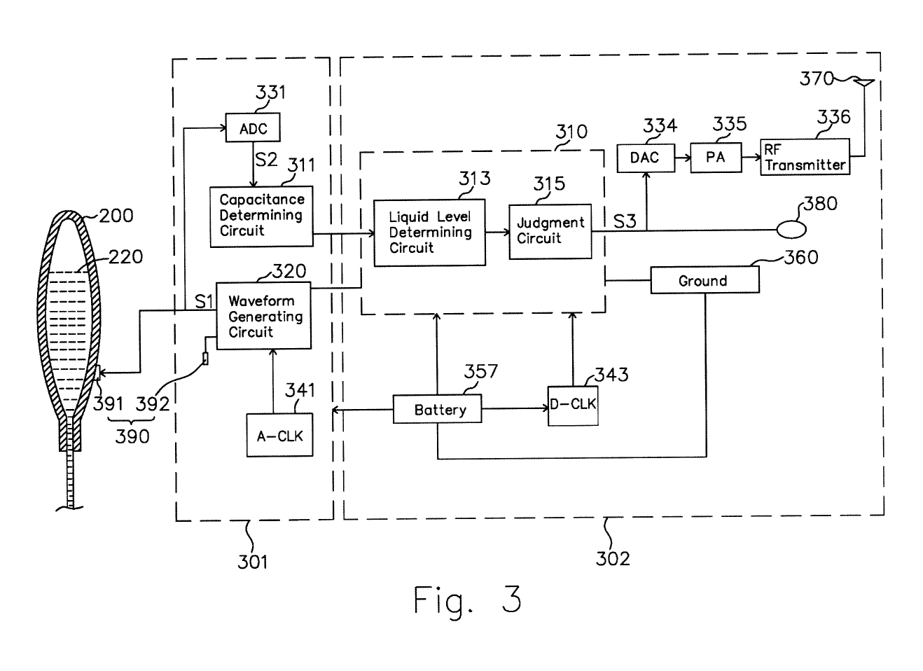

FIG. 3 is a block diagram of the detecting apparatus 300 of the first embodiment, wherein a liquid sensing module 301 is implemented in the case 395 of FIG. 1, and a processing and communication module 302 is implemented in the box 396. In operation, the liquid sensing module 301 determines the value of the capacitance of the capacitor 390 through the detecting electrode 391. The processing and communication module 302 converts the value of the capacitance of the capacitor 390 into the liquid level, and sends an alarm signal when the liquid level falls to the height of the detecting electrode 391. A battery 357 provides power to a waveform generating circuit 320 and a clock generator 341 of the liquid sensing module 301, and a processor 310 and another clock generator 343 of the processing and communication module 302.

More specifically, in the liquid sensing module 301, a waveform generating circuit 320 receives a clock signal from a clock generator 341 to generate a driving signal S1 of, e.g., a sine, square or triangular waveform. The driving signal S1 is an analog voltage signal, varying dependant on the value of the capacitance of the capacitor 390. The driving signal S1 is converted into a digital voltage signal S2 by an analog-to-digital converter 331 and sent to a capacitance determining circuit 311. The value of the capacitance of the capacitor 390 is determined by the capacitance determining circuit 311 and sent to a processor 310 of the processing and communication module 302.

In the processing and communication module 302, the clock generator 343 provides a clock signal for the operation of the processor 310. The processor 310 includes a liquid level determining circuit 313 and a judgment circuit 315, wherein the liquid level determining circuit 313 converts the value of the capacitance into the liquid level 220 in the container 200, and the judgment circuit 315 judges the liquid level 220 in the container 200 and sends an alarm signal S3 when the liquid level 220 falls to the height of the detecting electrode 391.

The alarm signal S3 can be sent out via wire or wireless transmission. In wire transmission, the alarm signal S3 is sent via output terminal 380. In wireless transmission, the alarm signal S3 is converted into an analog signal by a digital-to analog circuit 334, amplified by a power amplifier 335, and sent via a radio frequency (RF) transmitter 336 and an antenna 370.

Referring to FIGS. 1 and 3, the liquid sensing module 301 disposed in the case 395 includes the capacitance determining circuit 311, the waveform generating circuit 320, the analog-to-digital converter 331, the clock generator 341, and the grounding electrode 392. Note that the grounding electrode 392 in the case 395 is disposed adjacent to the waveform generating circuit 320, and electrically connected to ground 360 through the waveform generating circuit 320 and the processor 310. Such an arrangement provides a stable and accurate measurement of the liquid level.

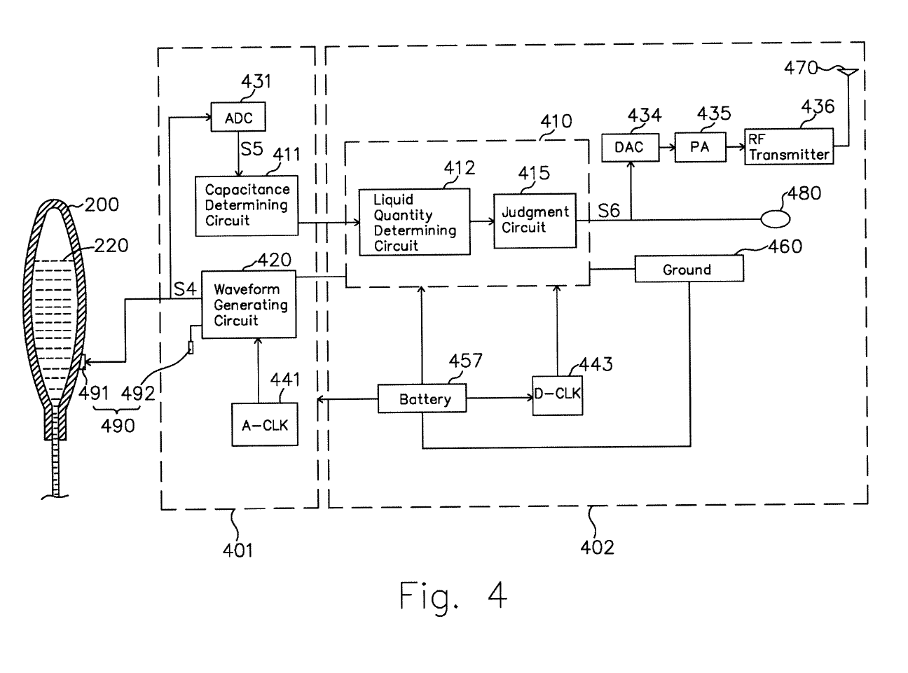

In a second embodiment, the remaining liquid quantity in an intravenous drip bag (or bottle or container) is detected. FIG. 4 is a block diagram of the detecting apparatus of the second embodiment, wherein a capacitor 490 includes a detecting electrode 491 attached to the outer surface of the intravenous drip bag 200 and a grounding electrode 492 spaced apart from the intravenous drip bag 200. In operation, a liquid sensing module 401 determines the value of the capacitance of the capacitor 490 through the detecting electrode 491. A processing and communication module 402 converts the value of the capacitance of the capacitor 490 into the remaining liquid quantity, and sends an alarm signal when the liquid level falls to the height of the detecting electrode 491. A battery 457 provides power to a waveform generating circuit 420 and a clock generator 441 of the liquid sensing module 401, and a processor 410 and another clock generator 443 of the processing and communication module 402.

More specifically, in the liquid sensing module 401, the waveform generating circuit 420 receives a clock signal from a clock generator 441 to generate a driving signal S4 of, e.g., a sine, square or triangular waveform. The driving signal S4 is an analog voltage signal, varying dependant on the value of the capacitance of the capacitor 490. The driving signal S4 is converted into a digital voltage signal S5 by an analog-to-digital converter 431 and sent to a capacitance determining circuit 411. The value of the capacitance of the capacitor 490 is determined by the capacitance determining circuit 411 and sent to a processor 410 of the processing and communication module 402.

In the processing and communication module 402, the clock generator 443 provides a clock signal for the operation of the processor 410. The processor 410 includes a liquid quantity determining circuit 412 and a judgment circuit 415, wherein the liquid quantity determining circuit 412 converts the value of the capacitance into the remaining liquid quantity in the intravenous drip bag 200, and the judgment circuit 415 judges the remaining liquid quantity and sends an alarm signal S6 when the liquid level 220 falls to the height of the detecting electrode 491.

TABLE 1

| Remaining Liquid | | Voltage |
|---|---|---|
| Quantity (ml) | Interval | (Stored Data) |
| 500-410 | I₆ | V₀ |
| | | V₀ + 2 |
| | | V₀ + 4 |
| | | V₀ + 6 |
| 409-320 | I₅ | V₁ |
| | | V₁ + 2 |
| | | V₁ + 4 |
| 319-230 | I₄ | V₂ |
| | | V₂ + 2 |
| 229-140 | I₃ | V₃ |
| | | V₃ + 2 |
| 139-50 | I₂ | V₄ |
| | | V₄ + 2 |
| | | V₄ + 4 |
| 49-0 | I₁ | V₅ |
| | | V₅ + 2 |
| | | V₅ + 4 |
| | | V₅ + 6 |
| | | V₆ |

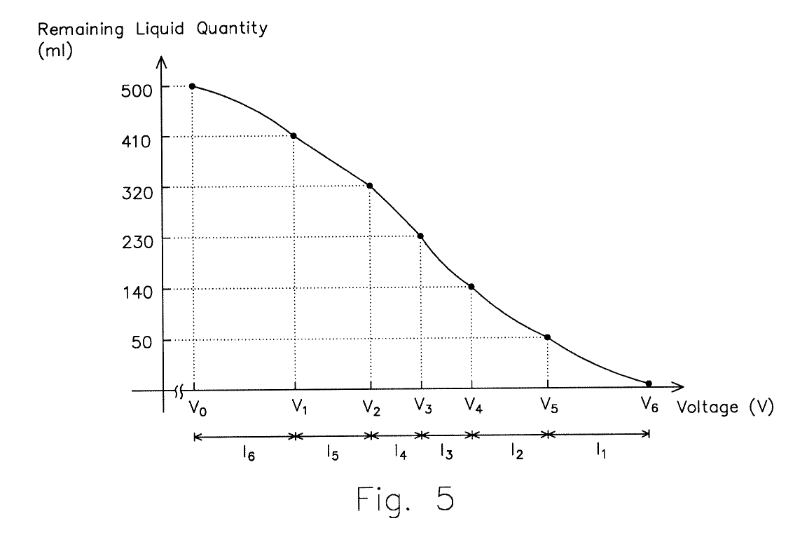

Table 1 is a lookup table of the digital voltage signal S5 and the remaining liquid quantity, which is measured in advance and stored in the liquid quantity determining circuit 412 for use during operation of the processor 410. The variation of the remaining liquid quantity in the intravenous drip bag 200 is divided into six intervals I₁-I₆. In the sixth interval I₆, for example, the remaining liquid quantity varies between 500-410 ml due to dripping, and the measured voltage signal S5 includes V₀ for 500 ml, V₀+2 for 470 ml, V₀+4 for 440 ml, and V₀+6 for 410 ml. Thus, the remaining liquid quantity at any time can be obtained by calculating the stored voltage data and, where appropriate, employing interpolation. For example, the obtained remaining liquid quantity is (500+470)/2 if the detected voltage signal is V₀+1. The relationship between the remaining liquid quantity and the voltage signal S5 is nonlinear, as shown in FIG. 5. Note that the six intervals I₁-I₆ are non-equidistant, wherein the side intervals are wider than the intermediate intervals. That is, I₆>I₅>I₄ and I₁>I₂>I₃.

As described above, an alarm signal S6 is sent out (or generated) by the processor 410 when the liquid level 220 falls to the height of the detecting electrode 491. The alarm signal S6 can be sent via wire or wireless transmission. In the case of wire transmission, the alarm signal S6 is sent via an output terminal 480. In the case of wireless transmission, the alarm signal S6 is converted into an analog signal by a digital-to-analog circuit 434, amplified by a power amplifier 435, and sent out by a radio frequency (RF) transmitter 436 and an antenna 470.

Similar to that of the first embodiment, the grounding electrode 492 is disposed adjacent to the waveform generating circuit 420 in an electrically isolated case, and electrically connected to ground 460 through the waveform generating circuit 420 and the processor 410. Such an arrangement provides a stable and accurate measurement of the remaining liquid quantity.

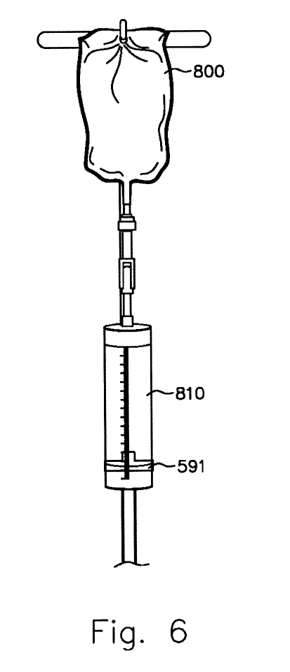

Referring to FIG. 6, in a third embodiment, a measuring cylinder 810 is disposed under an intravenous drip bag 800 for adding medicine to the liquid or solution. A detecting electrode 591 is attached to the lower portion of the measuring cylinder 810, detecting the remaining liquid quantity and dripping speed of the intravenous drip bag 800.

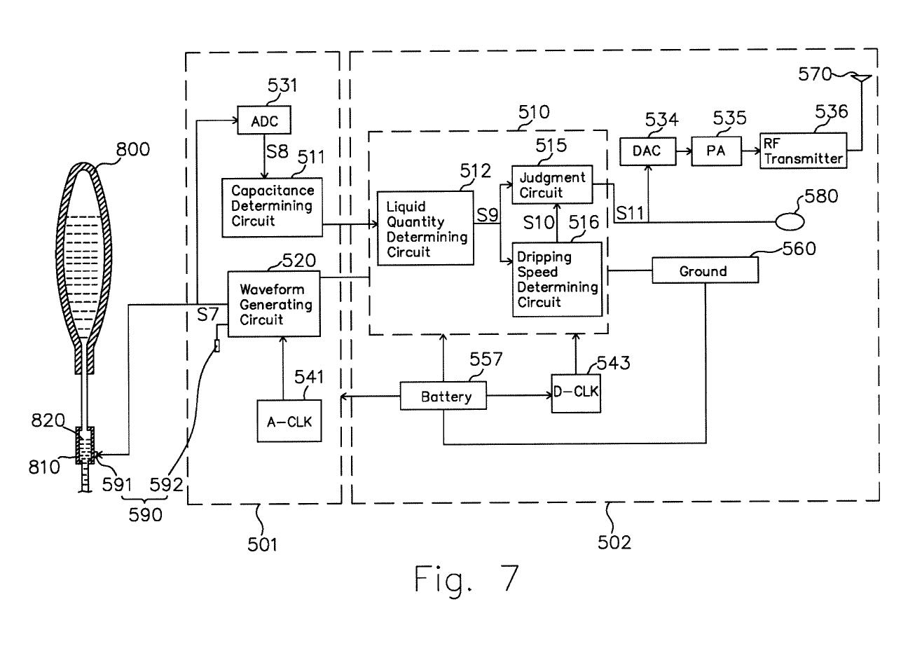

FIG. 7 is a block diagram of the detecting apparatus of the third embodiment, wherein a capacitor 590 includes a detecting electrode 591 attached to the measuring cylinder 810 and a grounding electrode 592 spaced apart from the measuring cylinder 810. In operation, a liquid sensing module 501 determines the value of the capacitance of the capacitor 590 through the detecting electrode 591. A processing and communication module 502 converts the value of the capacitance of the capacitor 590 into the remaining liquid quantity, calculates the dripping speed of the intravenous drip bag 800, and sends out an alarm signal when the liquid level falls to the height of the detecting electrode 591. A battery 557 provides power to a waveform generating circuit 520 and a clock generator 541 of the liquid sensing module 501, and a processor 510 and another clock generator 543 of the processing and communication module 502.

More specifically, in the liquid sensing module 501, the waveform generating circuit 520 receives a clock signal from a clock generator 541 to generate a driving signal S7 of, e.g., a sine, square or triangular waveform. The driving signal S7 is an analog voltage signal, varying dependant on the value of the capacitance of the capacitor 590. The driving signal S7 is converted into a digital voltage signal S8 by an analog-to-digital converter 531 and sent to a capacitance determining circuit 511. The value of the capacitance of the capacitor 590 is determined by the capacitance determining circuit 511 and sent to a processor 510.

In the processing and communication module 502, the clock generator 543 provides a clock signal for the operation of the processor 510. The processor 510 includes a liquid quantity determining circuit 512, a dripping speed determining circuit 516, and a judgment circuit 515. The liquid quantity determining circuit 512 converts the value of the capacitance into the remaining liquid quantity in the intravenous drip bag 800, and sends out a liquid quantity signal S9. The dripping speed determining circuit 516 receives the liquid quantity signal S9, calculates the dripping speed according to the variation of the liquid quantity, and sends a dripping speed signal S10. The judgment circuit 515 receives the dripping speed signal S10 from the dripping speed determining circuit 516 and sends it via wire or wireless transmission for, e.g., nurse's monitoring. Also, the judgment circuit 415 receives the liquid quantity signal S9 from the liquid quantity determining circuit 512 and sends an alarm signal S11 when the liquid level 820 falls to the height of the detecting electrode 591. The alarm signal S11 is sent via an output terminal 580. Alternatively, the alarm signal S11 is converted into an analog signal by a digital-to analog circuit 534, amplified by a power amplifier 535, and sent out by a radio frequency (RF) transmitter 536 and an antenna 570.

Similar to those of the first and second embodiments, the grounding electrode 592 is disposed adjacent to the waveform generating circuit 520 in an electrically isolated case, and electrically connected to ground 560 through the waveform generating circuit 520 and the processor 510.

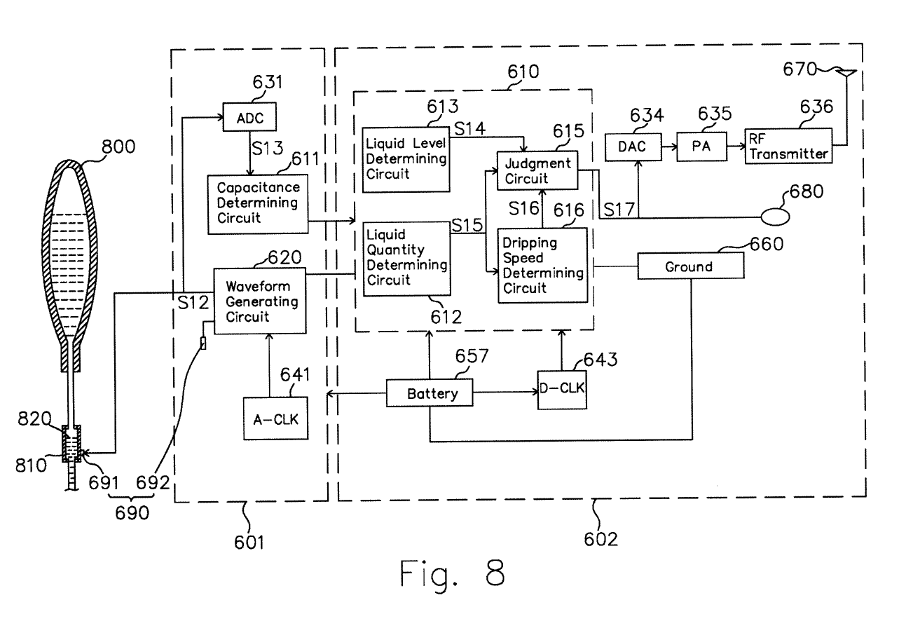

Referring to FIG. 8, in a fourth embodiment, a capacitor 690 includes a detecting electrode 691 and a grounding electrode 692. The detecting electrode 691 is attached to the lower portion of a measuring cylinder 810. A liquid sensing module 601 determines the value of the capacitance of the capacitor 690 through the detecting electrode 691. A processing and communication module 602 converts the value of the capacitance of the capacitor 690 into the liquid level and the remaining liquid quantity, calculates the dripping speed of the intravenous drip bag 800, and sends an alarm signal when the liquid level falls to the height of the detecting electrode 691. A battery 657 provides power to a waveform generating circuit 620 and a clock generator 641 of the liquid sensing module 601, and a processor 610 and another clock generator 643 of the processing and communication module 602.

More specifically, in the liquid sensing module 601, the waveform generating circuit 620 receives a clock signal from a clock generator 641 to generate a driving signal S12 of, e.g., a sine, square or triangular waveform. The driving signal S12 is an analog voltage signal, varying dependant on the value of the capacitance of the capacitor 690. The driving signal S12 is converted into a digital voltage signal S13 by an analog-to-digital converter 631 and sent to a capacitance determining circuit 611. The value of the capacitance of the capacitor 690 is determined by the capacitance determining circuit 611 and sent to a processor 610.

In the processing and communication module 602, the clock generator 643 provides a clock signal for the operation of the processor 610. The processor 610 includes a liquid quantity determining circuit 612, a liquid level determining circuit 613, a dripping speed determining circuit 616, and a judgment circuit 615. The liquid level determining circuit 613 converts the value of the capacitance into the liquid level in the intravenous drip bag 800, and sends a liquid level signal S14. The liquid quantity determining circuit 612 converts the value of the capacitance into the remaining liquid quantity in the intravenous drip bag 800, and sends a liquid quantity signal S15. The dripping speed determining circuit 616 receives the liquid quantity signal S15, calculates the dripping speed according to the variation of the liquid quantity, and sends a dripping speed signal S16. The judgment circuit 615 receives the dripping speed signal S16 from the dripping speed determining circuit 616 and sends it via wire or wireless transmission for, e.g., nurse's monitoring. Also, the judgment circuit 615 receives the liquid level signal S14 from the liquid level determining circuit 613 and the liquid quantity signal S15 from the liquid quantity determining circuit 612 and sends an alarm signal S17 when the liquid level 820 falls to the height of the detecting electrode 691. The alarm signal S17 is sent out via output terminal 680. Alternatively, the alarm signal S17 is converted into an analog signal by a digital-to-analog circuit 634, amplified by a power amplifier 635, and sent via a radio frequency (RF) transmitter 636 and an antenna 670.

Similar to those of the first, second and third embodiments, the grounding electrode 692 is disposed adjacent to the waveform generating circuit 620 in an electrically isolated case, and electrically connected to ground 660 through the waveform generating circuit 620 and the processor 610.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An apparatus for detecting a liquid level in an intravenous drip container, comprising:
a capacitor comprising a detecting electrode configured to be attached to an outer surface of the intravenous drip container, and a grounding electrode;
a clock generator configured to generate a clock signal;
a waveform generating circuit coupled to the detecting electrode configured to generate an analog driving signal by using the clock signal;
an analog-to-digital converter configured to convert the analog driving signal to a digital voltage signal;
a capacitance determining circuit configured to receive the digital voltage signal to determine a value of a capacitance of the capacitor; and
a processor configured to convert the value of the capacitance into the liquid level in the intravenous drip container, and send an alarm signal when the liquid level falls to a height of the detecting electrode,
wherein the grounding electrode is grounded through the waveform generating circuit and the processor.

2. The apparatus as claimed in claim 1, wherein the grounding electrode is disposed adjacent to the waveform generating circuit.

3. The apparatus as claimed in claim 1, wherein the processor comprises a liquid level determining circuit configured to convert the value of the capacitance into the liquid level in the intravenous drip container, and a judgment circuit configured to judge the liquid level and send the alarm signal when the liquid level falls to the height of the detecting electrode.

4. The apparatus as claimed in claim 1, further comprising a digital-to-analog circuit, a power amplifier, a radio frequency transmitter, and an antenna, wherein the alarm signal is converted from digital to analog by the digital-to-analog circuit, amplified by the power amplifier, and sent out by the radio frequency transmitter and the antenna.

5. The apparatus as claimed in claim 1, further comprising an output terminal configured to send the alarm signal.

6. The apparatus as claimed in claim 1, wherein the analog driving signal is a sine, square or triangular waveform.

7. An apparatus for detecting a remaining liquid quantity in an intravenous drip container, comprising:
a capacitor comprising a detecting electrode configured to be attached to an outer surface of the intravenous drip container, and a grounding electrode;
a clock generator generating a clock signal;
a waveform generating circuit coupled to the detecting electrode configured to generate an analog driving signal by using the clock signal;
an analog-to-digital converter configured to convert the analog driving signal to a digital voltage signal;
a capacitance determining circuit configured to receive the digital voltage signal to determine a value of a capacitance of the capacitor; and
a processor configured to convert the value of the capacitance into the remaining liquid quantity in the intravenous drip container, judge the remaining liquid quantity, and send an alarm signal when a liquid level in the intravenous drip container falls to a height of the detecting electrode,
wherein the grounding electrode is grounded through the waveform generating circuit and the processor.

8. The apparatus as claimed in claim 7, wherein the grounding electrode is disposed adjacent to the waveform generating circuit.

9. The apparatus as claimed in claim 7, wherein the processor comprises a liquid quantity determining circuit configured to convert the value of the capacitance into the remaining liquid quantity in the intravenous drip container, and a judgment circuit configured to judge the remaining liquid quantity and send the alarm signal when the liquid level falls to the height of the detecting electrode.

10. The apparatus as claimed in claim 9, wherein the liquid quantity determining circuit comprises a lookup table comprising non-equidistant intervals, by which the remaining liquid quantity is determined.

11. The apparatus as claimed in claim 7, further comprising a digital-to-analog circuit, a power amplifier, a radio frequency transmitter, and an antenna, wherein the alarm signal is converted from digital to analog by the digital-to-analog circuit, amplified by the power amplifier, and sent by the radio frequency transmitter and the antenna.

12. The apparatus as claimed in claim 7, further comprising an output terminal configured to send the alarm signal.

13. The apparatus as claimed in claim 7, wherein the analog driving signal is a sine, square or triangular waveform.

14. An apparatus for detecting a remaining liquid quantity and dripping speed of an intravenous drip container, comprising:
a capacitor comprising a detecting electrode configured to be attached to an outer surface of the intravenous drip container, and a grounding electrode;
a clock generator generating a clock signal;
a waveform generating circuit coupled to the detecting electrode configured to generate an analog driving signal by using the clock signal;
an analog-to-digital converter configured to convert the analog driving signal to a digital voltage signal;
a capacitance determining circuit configured to receive the digital voltage signal to determine a value of a capacitance of the capacitor; and
a processor configured to convert the value of the capacitance into the remaining liquid quantity in the intravenous drip container, determine the dripping speed by variation of the remaining liquid quantity, judge the remaining liquid quantity, and send an alarm signal when a liquid level in the intravenous drip container falls to a height of the detecting electrode,
wherein the grounding electrode is grounded through the waveform generating circuit and the processor.

15. The apparatus as claimed in claim 14, wherein the grounding electrode is disposed adjacent to the waveform generating circuit.

16. The apparatus as claimed in claim 14, wherein the processor comprises a liquid quantity determining circuit configured to convert the value of the capacitance into the remaining liquid quantity in the intravenous drip container, a dripping speed determining circuit configured to determine the dripping speed by the variation of the remaining liquid quantity, and a judgment circuit configured to judge the remaining liquid quantity and send the alarm signal when the liquid level falls to the height of the detecting electrode.

17. The apparatus as claimed in claim 16, wherein the liquid quantity determining circuit comprises a lookup table comprising non-equidistant intervals, by which the remaining liquid quantity is determined.

18. The apparatus as claimed in claim 14, further comprising a digital-to-analog circuit, a power amplifier, a radio frequency transmitter, and an antenna, wherein the alarm signal is converted from digital to analog by the digital-to-analog circuit, amplified by the power amplifier, and sent by the radio frequency transmitter and the antenna.

19. The apparatus as claimed in claim 14, further comprising an output terminal configured to send the alarm signal.

20. The apparatus as claimed in claim 14, wherein the analog driving signal is a sine, square of triangle waveform.

21. An apparatus for detecting a liquid in an intravenous drip container, comprising:
a detecting electrode configured to be attached to an outer surface of the intravenous drip container;
a liquid sensing module which comprises a clock generator generating a clock signal, a waveform generating circuit coupled to the detecting electrode configured to generate an analog driving signal by using the clock signal, a grounding electrode grounded through the waveform generating circuit, an analog-to-digital converter configured to convert the analog driving signal to a digital voltage signal, and a capacitance determining circuit configured to receive the digital voltage signal to determine a value of a capacitance of a capacitor formed by the detecting electrode and the grounding electrode; and
a connecting means connecting the detecting electrode to the liquid sensing module.

22. The apparatus as claimed in claim 21, wherein the connecting means is a clip.

23. The apparatus as claimed in claim 22, wherein the detecting electrode is T-shaped and comprises a bottom portion configured to be attached to the outer surface of the intravenous drip container, and a top portion which is flexible to be held by the clip.

24. The apparatus as claimed in claim 21, further comprising an electrically isolated case in which the liquid sensing module is disposed.

25. The apparatus as claimed in claim 21, further comprising a processing and communication module which converts the value of the capacitance into a liquid level in the intravenous drip container, and sends an alarm signal when the liquid level falls to a height of the detecting electrode.

26. The apparatus as claimed in claim 21, further comprising a processing and communication module which converts the value of the capacitance into a remaining liquid quantity in the intravenous drip container, judges the remaining liquid quantity, and sends an alarm signal when a liquid level in the intravenous drip container falls to a height of the detecting electrode.

27. The apparatus as claimed in claim 21, further comprising a processing and communication module which converts the value of the capacitance into a remaining liquid quantity in the intravenous drip container, determines a dripping speed by variation of the remaining liquid quantity, judges the remaining liquid quantity, and sends an alarm signal when a liquid level in the intravenous drip container falls to a height of the detecting electrode.
